SEQUENCE LISTING (7 sequences)

SEQ ID NO: 1 - DNA, 1797 bp, Artificial Sequence
  hM3Dq polynucleotide sequence
atgaccttgcacaataacagtacaacctcgcctttgtttccaaacatcagctcctcctggatacacagcccctccgatgcagggctgcccccgggaaccgtcactcatttcggcagctacaatgtttctcgagcagctggcaatttctcctctccagacggtaccaccgatgaccctctgggaggtcataccgtctggcaagtggtcttcatcgctttcttaacgggcatcctggccttggtgaccatcatcggcaacatcctggtaattgtgtcatttaaggtcaacaagcagctgaagacggtcaacaactacttcctcttaagcctggcctgtgccgatctgattatcggggtcatttcaatgaatctgtttacgacctacatcatcatgaatcgatgggccttagggaacttggcctgtgacctctggcttgccattgactgcgtagccagcaatgcctctgttatgaatcttctggtcatcagctttgacagatacttttccatcacgaggccgctcacgtaccgagccaaacgaacaacaaagagagccggtgtgatgatcggtctggcttgggtcatctcctttgtcctttgggctcctgccatcttgttctggcaatactttgttggaaagagaactgtgcctccgggagagtgcttcattcagttcctcagtgagcccaccattacttttggcacagccatcgctggtttttatatgcctgtcaccattatgactattttatactggaggatctataaggaaactgaaaagcgtaccaaagagcttgctggcctgcaagcctctgggacagaggcagagacagaaaactttgtccaccccacgggcagttctcgaagctgcagcagttacgaacttcaacagcaaagcatgaaacgctccaacaggaggaagtatggccgctgccacttctggttcacaaccaagagctggaaacccagctccgagcagatggaccaagaccacagcagcagtgacagttggaacaacaatgatgctgctgcctccctggagaactccgcctcctccgacgaggaggacattggctccgagacgagagccatctactccatcgtgctcaagcttccgggtcacagcaccatcctcaactccaccaagttaccctcatcggacaacctgcaggtgcctgaggaggagctggggatggtggacttggagaggaaagccgacaagctgcaggcccagaagagcgtggacgatggaggcagttttccaaaaagcttctccaagcttcccatccagctagagtcagccgtggacacagctaagacttctgacgtcaactcctcagtgggtaagagcacggccactctacctctgtccttcaaggaagccactctggccaagaggtttgctctgaagaccagaagtcagatcactaagcggaaaaggatgtccctggtcaaggagaagaaagcggcccagaccctcagtgcgatcttgcttgccttcatcatcacttggaccccatacaacatcatggttctggtgaacaccttttgtgacagctgcatacccaaaaccttttggaatctgggctactggctgtgctacatcaacagcaccgtgaaccccgtgtgctatgctctgtgcaacaaaacattcagaaccactttcaagatgctgctgctgtgccagtgtgacaaaaaaaagaggcgcaagcagcagtaccagcagagacagtcggtcatttttcacaagcgcgcacccgagcaggccttgaaggatcccccggtcgccaccatgtaa

SEQ ID NO: 2 - DNA, 1464 bp, Artificial Sequence
  hM4Di polynucleotide sequence
atggccaacttcacacctgtcaatggcagctcgggcaatcagtccgtgcgcctggtcacgtcatcatcccacaatcgctatgagacggtggaaatggtcttcattgccacagtgacaggctccctgagcctggtgactgtcgtgggcaacatcctggtgatgctgtccatcaaggtcaacaggcagctgcagacagtcaacaactacttcctcttcagcctggcgtgtgctgatctcatcataggcgccttctccatgaacctctacaccgtgtacatcatcaagggctactggcccctgggcgccgtggtctgcgacctgtggctggccctggactgcgtggtgagcaacgcctccgtcatgaaccttctcatcatcagctttgaccgctacttctgcgtcaccaagcctctcacctaccctgcccggcgcaccaccaagatggcaggcctcatgattgctgctgcctgggtactgtccttcgtgctctgggcgcctgccatcttgttctggcagtttgtggtgggtaagcggacggtgcccgacaaccagtgcttcatccagttcctgtccaacccagcagtgacctttggcacagccattgctggcttctacctgcctgtggtcatcatgacggtgctgtacatccacatctccctggccagtcgcagccgagtccacaagcaccggcccgagggcccgaaggagaagaaagccaagacgctggccttcctcaagagcccactaatgaagcagagcgtcaagaagcccccgcccggggaggccgcccgggaggagctgcgcaatggcaagctggaggaggcccccccgccagcgctgccaccgccaccgcgccccgtggctgataaggacacttccaatgagtccagctcaggcagtgccacccagaacaccaaggaacgcccagccacagagctgtccaccacagaggccaccacgcccgccatgcccgcccctcccctgcagccgcgggccctcaacccagcctccagatggtccaagatccagattgtgacgaagcagacaggcaatgagtgtgtgacagccattgagattgtgcctgccacgccggctggcatgcgccctgcggccaacgtggcccgcaagttcgccagcatcgctcgcaaccaggtgcgcaagaagcggcagatggcggcccgggagcgcaaagtgacacgaacgatctttgccattctgctggccttcatcctcacctggacgccctacaacgtcatggtcctggtgaacaccttctgccagagctgcatccctgacacggtgtggtccattggctactggctctgctacgtcaacagcaccatcaaccctgcctgctatgctctgtgcaacgccacctttaaaaagaccttccggcacctgctgctgtgccagtatcggaacatcggcactgccaggcgggatccaccggtcgccaccatgtaa

SEQ ID NO: 3 - DNA, 34 bp, Artificial Sequence
  LoxP polynucleotide sequence
ataacttcgtatagcatacattatacgaagttat

SEQ ID NO: 4 - DNA, 34 bp, Artificial Sequence
  LoxP reverse oriented polynucleotide sequence
ataacttcgtataatgtatgctatacgaagttat

SEQ ID NO: 5 - DNA, 34 bp, Artificial Sequence
  Lox2272 polynucleotide sequence
ataacttcgtataggatactttatacgaagttat

SEQ ID NO: 6 - DNA, 34 bp, Artificial Sequence
  Lox2272 reverse oriented polynucleotide sequence
ataacttcgtataaagtatcctatacgaagttat

SEQ ID NO: 7 - DNA, 1011 bp, Artificial Sequence
  Cre recombinase polynucleotide sequence
atggccaatttactgaccgtacaccaaaatttgcctgcattgccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgtaa

(19) United States
(12) Patent Application Publication
(10) Pub. No.: US 2023/0000064 A1
(43) Pub. Date: Jan. 5, 2023

(54) OBSESSIVE-COMPULSIVE DISORDER ANIMAL MODEL AND PRODUCTION METHOD THEREOF

(71) Applicant: KOREA UNIVERSITY RESEARCH AND BUSINESS FOUNDATION, Seoul (KR)

(72) Inventors: Bong June YOON, Seoul (KR); In Bum LEE, Seoul (KR)

(73) Assignee: KOREA UNIVERSITY RESEARCH AND BUSINESS FOUNDATION, Seoul (KR)

(21) Appl. No.: 17/784,467

(22) PCT Filed: Nov. 26, 2020
(86) PCT No.: PCT/KR2020/016994

§ 371 Date: Jun. 10, 2022

(30) Foreign Application Priority Data
Dec. 13, 2019 (KR) 10-2019-0166845

(51) Int. Cl. A01K 67/027; C12N 15/85

(52) U.S. Cl. CPC A01K 67/0275; C12N 15/8509; A01K 2267/0356; C12N 2015/8527; C12N 2750/14141

(57) ABSTRACT

Provided is an obsessive-compulsive disorder animal model and a production method thereof, in which a neuronal circuit connecting the basolateral amygdala (BLA) and the dorsomedial striatum (DMS) is activated. In the present disclosure, it was confirmed that the BLA and DMS are connected to each other, and that when the BLA-DMS neuronal circuit is activated, there is a large increase in checking, repeating, cleaning, and collecting, which are compulsive behaviors representative of obsessive-compulsive disorder, and a decrease in cognitive flexibility. Therefore, animals in which the BLA-DMS neuronal circuit is activated may be useful as animal models for studying obsessive-compulsive disorder. In particular, the obsessive-compulsive disorder animal model can reproduce all of the compulsive behaviors and thus may become the first animal model to provide an understanding of the interactions between anxiety behaviors and compulsive behaviors, which existing animal models have not been able to provide.

### TECHNICAL FIELD

The present disclosure relates to an obsessive-compulsive disorder animal model and a construction method therefor and, more specifically, to an obsessive-compulsive disorder animal model wherein a neural circuit connecting from the basolateral amygdala to the dorsomedial striatum is activated and a construction method therefor.

### BACKGROUND ART

The orbitofrontal cortex (OFC) is a site of the brain which has been solely known to be associated with obsessive compulsive disorder (hereinafter abbreviated as “OCD”), thus far. Inter alia, the activation of the medial orbitofrontal cortex (mOFC) causes OCD (Repeated cortico-striatal stimulation generates persistent OCD-like behavior. Science (New York, N.Y.), 340(6137), 1234-1239. (2013)) while the activation of the lateral orbitofrontal cortex (1° F.C) is suppressive of OCD (Optogenetic stimulation of lateral orbitofronto-striatal pathway suppresses compulsive behaviors. Science (New York, N.Y.), 340(6137), 1243-1246. (2013)).

OCD is one of the very high rates of mental illness with a prevalence of 2%-3% worldwide. OCD is characterized by a severe obsession that appears with a particular repetitive convention (compulsion). Obsessive compulsive behaviors are common behaviors that occur in a similar pattern not only in patients diagnosed with OCD, but also in Tourette syndrome, drug addiction, and compulsive eating disorder, and are a major symptom involved in various mental disorders. However, there has not been any suggestion of neurological basis for the existence of an interaction between anxiety behavior and OCD. Currently, the first-line treatment relies on selective serotonin reuptake inhibitor (SSRI)-based drugs that act on the representative neurotransmitter serotonin system. In order to understand various related diseases and advance therapeutic techniques, there is therefore a need for development of a new animal model that helps understand the molecular and neuroscientific mechanisms of compulsions and simultaneously exhibits other OCD patterns in addition to specific compulsions (mainly grooming).

Given the current situation that a significant number of patients are resistant to currently available treatments (mainly SSRI-based), the development of new therapeutic techniques can be expected to have a very high ripple effect. Therefore, providing an integrated animal model of OCD is expected to be very useful in understanding the mechanisms of OCD as well as several related diseases (such as Tourette syndrome, adduction, comprehensive eating disorder, etc.) and in developing therapeutic and diagnostic techniques.

Existing OCD-related animal models have the disadvantage of not being able to reproduce all the various characteristics of obsessive-compulsive disorder because they are constructed by mainly deleting specific genes.

By contrast, the model suggested by the present disclosure is designed to alter the activity of the basal ganglia circuitry and expected to provide understanding of the mechanism of OCD in view of neural circuits. In particular, the model of the present disclosure will be the first animal model to provide an understanding of the interaction between anxiety behavior and obsessive-compulsive behavior that conventional models have not provided.

Leading to the present disclosure, intensive and thorough research, conducted by the present inventors under the background, into elucidating the neural circuit site closely related to the mechanism of OCD, resulted in the finding that there is connectivity from the basolateral amygdala (hereinafter abbreviated as “BLA”) to the dorsomedial striatum (hereinafter abbreviated as “DMS”) and activation of the BLA-DMS neural circuit provokes representative obsessive-compulsive behaviors of OCD, such as checking, repetitive, cleaning, or hoarding behaviors.

### DISCLOSURE OF INVENTION

### Technical Problem

Therefore, an aspect of the present disclosure is to provide a method for constructing an animal model of obsessive-compulsive disorder that can reproduce all comprehensive obsessive-compulsive behaviors (checking, repetitive, cleaning, and hoarding behaviors).

Another aspect of the present disclosure is to provide an animal model of obsessive-compulsive disorder, constructed by the method, which reproduces comprehensive obsessive-compulsive behaviors.

A further aspect of the present disclosure is to provide a method for screening a candidate drug for prevention or treatment of obsessive-compulsive disorder by using the animal model of obsessive-compulsive disorder that reproduces comprehensive obsessive-compulsive behaviors.

### Solution to Problem

To achieve the aims, the present disclosure provides a method for constructing an animal model of obsessive-compulsive disorder, the method comprising a step of activating a neural circuit connecting from basolateral amygdala to dorsomedial striatum in animals exclusive of humans.

In an embodiment of the present disclosure, the neural circuit may be activated through physical activation or chemical activation.

In an embodiment of the present disclosure, the physical activation may be carried out by injecting a virus carrying a channel rhodopsin gene into a basolateral amygdala site, installing an optical fiber in a dorsomedial striatum site, and applying light to the dorsomedial striatum site to activate the neural circuit.

In an embodiment of the present disclosure, the chemical activation may be carried out by injecting a virus carrying a double-floxed inverted open reading frame (DIO) and a chemogenetic protein gene to a basolateral amygdala site, injecting a retrograde virus carrying a Cre recombinase gene to a dorsomedial striatum site, and then applying a drug upon activation of the viruses to active the neural circuit.

In an embodiment of the present disclosure, the virus may be adeno-associated virus (AAV).

In an embodiment of the present disclosure, the chemogenetic protein may be selected from the group consisting of hM2Di, hM4Di, hM3Dq, and hM5Dq.

In an embodiment of the present disclosure, the drug activates a chemogenetic protein and may be selected from the group consisting of clozapine N-oxide, clozapine, compound 21, and perlapine.

Also, the present disclosure provides an animal model of obsessive-compulsive disorder, constructed by the method.

In an embodiment of the present disclosure, the animal model may exhibit all of checking behavior, repetitive behavior, cleaning behavior, and hoarding behavior.

In addition, the present disclosure provides a method for screening a candidate drug for prevention or treatment of obsessive-compulsive disorder, the method comprising the steps of: administering a candidate drug to the animal model of obsessive-compulsive disorder; and determining whether any of obsessive-compulsive disorder-caused behaviors including checking behavior, repetitive behavior, cleaning behavior, and hoarding behavior is reduced.

### Advantageous Effects of Invention

In the present disclosure, it is confirmed that the BLA and DMS are connected to each other and that the activation of the BLA-DMS neuronal circuit leads to a remarkable increase in the frequencies of conducing checking, repetitive, cleaning, and hoarding behaviors, which are compulsive behaviors representative of obsessive-compulsive disorder, and a decrease in cognitive flexibility. Therefore, animals in which the BLA-DMS neural circuit is activated may be useful as animal models for studying obsessive-compulsive disorder. In particular, the animal model of obsessive-compulsive disorder according to the present disclosure can reproduce all of the compulsive behaviors (checking, repetitive, cleaning, and hoarding behaviors), and thus may become the first animal model to provide an understanding of the interaction between anxiety behaviors and compulsive behaviors, which has not yet been provided by existing animal models.

### BRIEF DESCRIPTION OF DRAWINGS

FIG. 1 shows connectivity between the basolateral amygdala (BLA) and the dorsomedial striatum (DMS) among subunits of the brain, as assayed by antegrade staining with AAV-tdTomato and retrograde staining with CTB (choleratoxin subunit b). 1a: a schematic diagram of a process of injecting AAV-tdTomato to a BLA site; 1b and 1c: a striatum site stained with the injection of AAV-tdTomato to a BLA site. 1d: a schematic diagram of a process of injecting CTB into a DMS site; and 1e and 1f: a BLA site stained with the injection of CTB to a DMS site.

FIG. 2 is a schematic diagram of a process of injecting AAV-ChR2 virus to a BLA site and installing an optical fiber a DMS site in a mouse.

FIG. 3 shows results of an elevated plus maze (EPM) test for evaluating degrees of anxiety after activating the BLA-DMS neural circuit through an Optogenetics technique. The linear plot in the left panel shows grooming duration in the presence or absence of the light applied to the mice and the bar graph in the right panel shows times spent at the center, the closed arm, and open arms in the elevated plus maze in the presence or absence of the light applied to the mice.

FIG. 4 shows a square acrylic plate 30 cm wide and 30 cm long with 9 holes (3 cm in diameter), designed for a hole beard test. In the hole board of the present disclosure, a home base formed of a material different from the acrylic plate was established, and based on the home base, water was placed on the right end of the acrylic plate and 10% sucrose was placed on the other side.

FIG. 5 is a schematic diagram showing a process for constructing an animal model of obsessive-compulsive disorder in time series.

FIG. 6 is a plot of frequencies of checking behavior with time in a hole board test using animal models of obsessive-compulsive disorder. Sucrose SAL: frequency of heading toward 10% sucrose in saline-administered group; Sucrose CNO: frequency of heading toward 10% sucrose in Clozapine n-oxide-administered group; Water SAL: frequency of heading toward water in saline-administered group; and Water CNO: frequency of heading toward water in Clozapine n-oxide-administered group.

FIG. 7 is a plot of frequencies of a behavior of repetitively inserting the nose into the holes (nose poking) with time in a hole board test using animal models of obsessive-compulsive disorder (SAL: saline-administered group, CNO: Clozapine n-oxide-administered group).

FIG. 8 shows plots of grooming duration and frequency with time in animal models of obsessive-compulsive disorder (SAL: saline-administered group, CNO: Clozapine n-oxide-administered group).

FIG. 9 is a plot of a total amount of beverage consumed with time in animal models of obsessive-compulsive disorder. Sucrose SAL: amount of 10% sucrose consumed by saline-administered group, Sucrose CNO: amount of 10% sucrose consumed by Clozapine n-oxide-administered group, Water SAL: amount of water consumed by saline-administered group, Water CNO: amount of water consumed by Clozapine n-oxide-administered group.

FIG. 10 shows results of a cognitive flexibility test in animal models of obsessive-compulsive disorder (SAL: saline-administered group, CNO: Clozapine n-oxide-administered group).

FIG. 11 is a photographic image of a tool designed to examine hoarding patterns in animal models of obsessive-compulsive disorder. Two cages were attached to each other with a central passageway. In order to allow the mice to recognize one case as a home case, the passageway to the opposite side were blocked for one day (day 12). The next day (day 13), food and toys were placed on the opposite side and the blocked passage was opened.

FIG. 12 shows hoarding patterns of a CNO (Clozapine n-oxide)-administered group in animal models of obsessive-compulsive disorder.

FIG. 13 shows hoarding patterns of a SAL (saline)-administered group in animal models of obsessive-compulsive disorder.

FIG. 14 shows quantified hoarding patterns of food and toys between CNO (Clozapine n-oxide)- and SAL (saline)-administered groups in animal models of obsessive-compulsive disorder.

FIG. 15 shows photographic images of hairs around mouse mouths of CNO (Clozapine n-oxide)- and SAL (saline)-administered groups in animal models of obsessive-compulsive disorder. Due to excessive grooming, the hairs around the mouth of the Clozapine n-oxide (CNO)-administered mice were observed to disappear after 20 days from the last test day.

### BEST MODE FOR CARRYING OUT THE INVENTION

The present disclosure pertains to a method for constructing an animal model of obsessive-compulsive disorder and, more specifically, to a method for constructing an animal model of obsessive-compulsive disorder, the method comprising a step of activating a neural circuit connecting between the basolateral amygdala and the dorsomedial striatum in an animal.

As used herein, “obsessive-compulsive disorder (OCD)” is a subtype of anxiety disorder in which unwanted thoughts and actions are repeated, and is known to have the main symptoms of obsessions that are persistent painful thoughts, impulses, or images recurring to the consciousness, and compulsions that are repetitive actions to mitigate anxiety. Compulsive behaviors appear in the form of cleaning behavior, checking behavior, repetitive behavior, arranging behavior, delaying behavior, etc., and patients with OCD repeat the behaviors to recover from the anxiety that stems from obsessive thoughts, even though they know it is inappropriate and excessive.

As used herein, the “amygdala” is a part of the limbic system in the brain and performs a primary role in the processing of information pertaining to motivation, learning, and emotional responses. It is an organ consisting of 10 or more nuclei classified largely into three parts including basolateral nuclei, corticomedial nuclei, and central nuclei and receives information from various regions. First, the information received from sensory organs of the body is sent to the basolateral nuclei of the amygdala and then transferred to the cerebral cortex to make emotional experiences. Among sensory signals, olfactory signals penetrate into the corticomedial nuclei of the amygdala. The sensory signals introduced into the amygdala are connected to the central nuclei and then sent to the autonomic nervous system. The signals are in turn transferred to the hypothalamus to generate physiological responses such as stress hormone release or awakening.

The term “basolateral amygdala” (BLA), as used herein, refers to an area located at the basolateral side (basal side) of the amygdala.

As used herein, the term “striatum” refers to an area of the basal ganglia of the brain that plays an important role in the selection and initiation of movement through neural network connections with the cerebral cortex and thalamus. In primates, the striatum is divided into a ventral striatum and a dorsal striatum, subdivisions that are based upon function and connections. The ventral striatum consists of the nucleus accumbens (NAc) and the olfactory tubercle. The dorsal striatum consists of the caudate nucleus and the putamen.

As used herein, the term “dorsomedial striatum” (DMS) refers to an area that is located at the dorsal medial side of the striatum and corresponds to the caudate nucleus in primates.

In the present disclosure, it was found that there is connectivity from the basolateral amygdala (BLA) to the dorsomedial striatum (DMS) and activation of the BLA-DMS neural circuit provokes representative compulsive behaviors of obsessive-compulsive disorder, such as checking behavior, repetitive behavior, cleaning behavior, or hoarding behavior, as analyzed through experiments.

Above all, it was found that there is a neural circuit connecting from BLA to DMS.

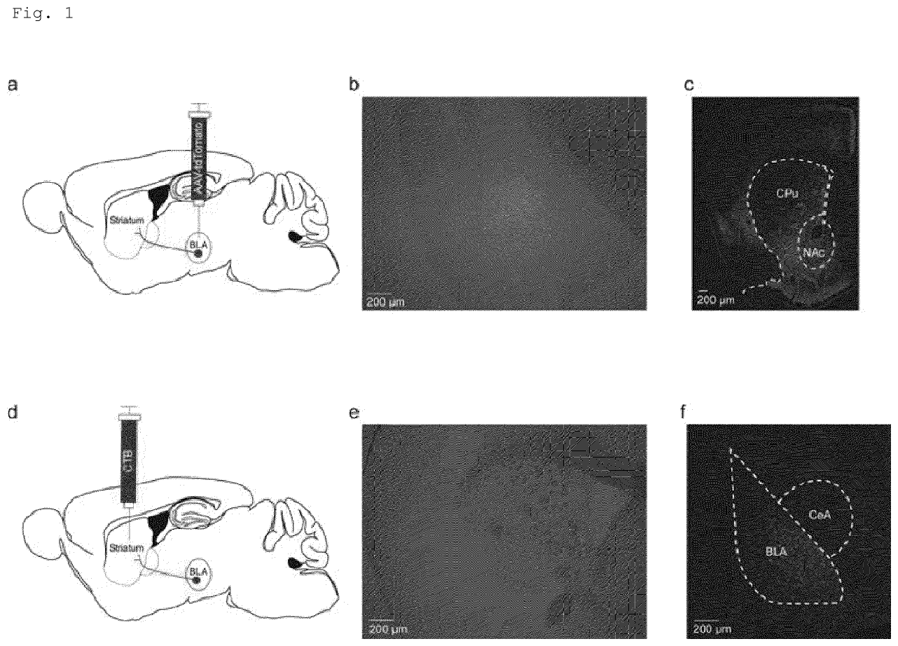

In detail, BLA, when injected with AAV-tdTomato, was stained red in the forward direction. At this location, a striatum area represented as the caudate-putamen (CPu), inter alia, a dorsomedial striatum (DMS) area and a ventral striatum area (NAc) were stained. After choleratoxin subunit b (CTB) was injected into a target area of DMS, staining in the reverse direction toward the BLA was detected, revealing that there is a circuit from the BLA to the DMS (see FIG. 1).

This BLA-DMS neural circuit has not yet been known previously.

In the present disclosure, optogenetics experiments were conducted to identify the role of the BLA-DMS neural circuit. When the BLA-DMS neural circuit is activated, grooming activity, which is a representative behavior of OCD, becomes excessive. Grooming in mice is an action corresponding to the repetitive hand wash known as a representative symptom of obsessive-compulsive disorder.

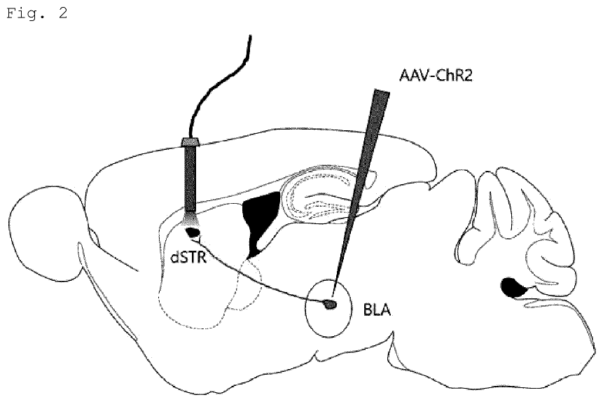

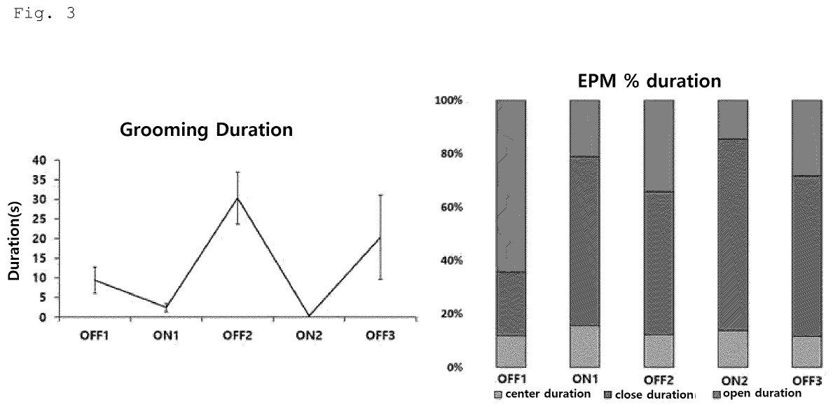

For greater detail, AAV-ChR2 was injected into the BLA and an optical fiber was installed in the DMS, followed by exposure of the DMS to a blue radiation (473 nm) to specifically activate the BLA-DMS neural circuit, under the condition of which an EPM (elevated plus-maze) test was conducted (see FIG. 2). When AAV-ChR2 is injected into the BLA, the axons projecting from the BLA express channelrhodopsin (ChR2) at the ends thereof. When absorbing blue light with a wavelength of 470 nm, the channelrhodopsin (ChR2), located at the cell membrane, allows the inflow of Na⁺ ions into cells to activate neurons. In an embodiment of the present disclosure, blue light (473 nm) was emitted to the optical fiber installed in the DMS to activate the BLA-DMS neural circuit. The activation of the BLA-DMS neural circuit was observed to increase anxiety, leading to the onset of obsessive-compulsive disorder (see FIG. 3).

In addition, an animal model of obsessive-compulsive disorder was constructed using AAV virus and the chemogenetic technique designer receptor exclusively activated by designer drug (DREADD) system in the present disclosure in order to activate the BLA-DMS circuit in a long term, not temporarily. The mice in which the BLA-DMS neural circuit is activated for a long term were observed to exhibit representative compulsive behaviors of obsessive-compulsive disorder, such as checking behavior, repetitive behavior, cleaning behavior, and hoarding behavior.

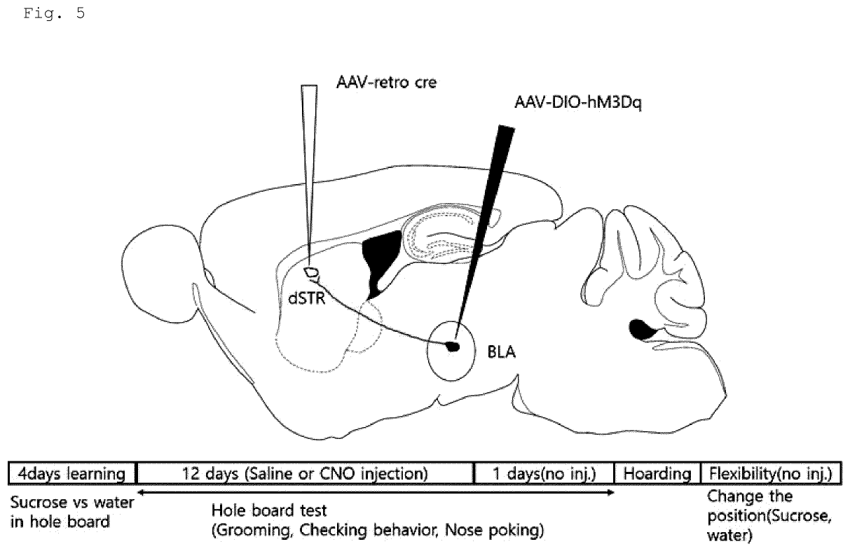

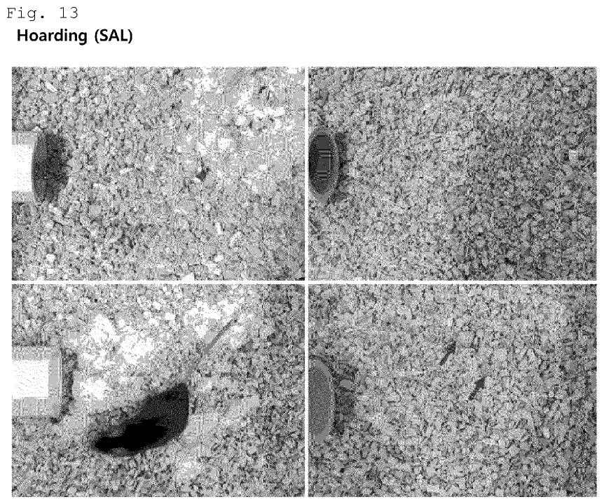

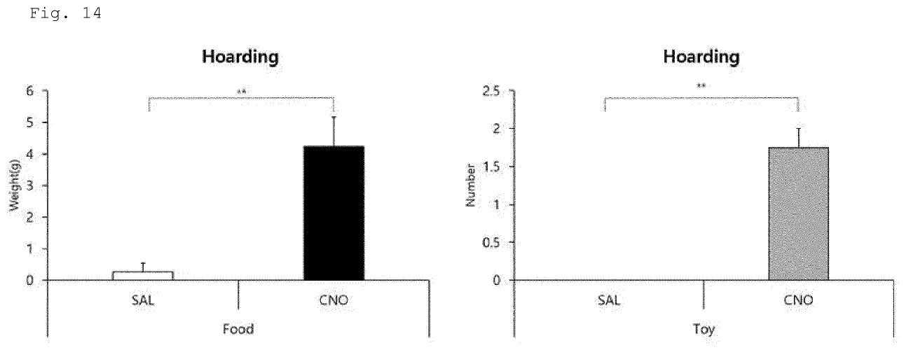

In greater detail, retrograde AAV carrying a gene coding for a Cre recombinant enzyme was injected into an area of the DMS and AAV-DIO-hM3Dq virus specifically expressing cre was injected to an area of the BLA, after which when the introduced virus was activated, a specific drug was administered to specifically the BLA-DMS neural circuit. For reference, this is based on the principle that when the retrograde virus from the DMS expresses Cre in the neuron cell body (soma) of the BLA, the AAV-DIO-hM3Dq virus is induced to express the target gene (hM3Dq) through the Double-Floxed Inverted Open reading frame (DIO) only in the presence of Cre. hM3Dq can activate neurons in cooperation with the membrane protein GPCR (G-protein-coupled receptors) upon stimulation with a specific drug, whereby the specific activation of the BLA-DMS neural circuit can be achieved. For mice adapted to activate the BLA-DMS neural circuit for a long term, the checking behavior, repetitive behavior, cleaning behavior, and hoarding behavior, which correspond to the representative compulsive behaviors of obsessive-compulsory disorder, were significantly increased while cognitive flexibility decreased (see FIGS. 5 to 15).

Herein, “adeno-associated virus” (AAV), which is a type of parvovirus, is a non-pathogenic virus that does not provoke a significant immune response and has a broad spectrum of host cells. AAV can infect both dividing and quiescent cells. Having ability to integrate virally carried genes into the host genome in addition to existing in the form of an episomal DNA within host cells, AAV is available as a gene carrier (vector) for gene therapy and thus has recently attracted considerable interest. Particularly when infecting human cells, the virus tends to integrate the viral genome into a specific site in the human chromosome 19. Since this site is known to be free of genes responsible for special functions, the insertional mutagenesis-induced activation of oncogenes is highly unlikely to occur. Hence, AAV is further advantageous for gene therapy. Moreover, chromosomal insertion is almost not carried out by adeno-associated viruses in a recombinant form, and the reason remains unclear yet.

Due to the aforementioned advantages, adeno-related virus (AAV) has been considered as a gene carrier (vector) that can introduce foreign genes necessary for treatment into a subject to be treated. Indeed, it has been reported that AAV is likely to be used as an important carrier for obtaining therapeutic effects in various diseases.

However, adeno-associated virus (AAV) is difficult to effectively prepare into recombinant AAV (rAAV) in an amount necessary for treatment because it lacks an independent self-replication ability. Due to this disadvantage, recombinant AAV has not been widely used since the early gene therapy research, but as a result of steady research on the life cycle of viruses, effective methods for the production of recombinant AAV (rAAV), such as helper virus-free production systems, hybrid helper virus systems, or packaging cell lines transformed with essential adenovirus and/or AAV genes, have been developed.

The most widely used among these methods is double or triple transduction of a comprehensive helper plasmid covering a recombinant AAV (rAAV) expression vector carrying a target gene, an AAV rep-cap gene expression vector (e.g., pAAV-RC), and an expression vector harboring a gene of adenovirus origin necessary for forming infectious particles (e.g., pHelper) (that is, a plasmid carrying both an AAV rep-cap gene and a gene of adenovirus origin necessary for forming infectious adeno-associated virus particles, i.e., pDG) into an adenovirus E1 gene-transformed packaging cell line, such as HEK293 cells.

In most cases, transduction is generally performed in an adherent culture of a packaging cell line capable of being supplemented with the E1 gene of adenovirus, such as human embryonic kidney 293 cells (HEK 293).

As used herein, the term “retrograde virus” refers to a recombinant AAV (rAAV) variant that mediates a retrograde access to projection neurons and is capable of retrograde transport from axon to its soma in a neural circuit.

In the present disclosure, an AAV helper-free system was used for constructing an animal model of obsessive-compulsive disorder. In greater detail, advantage was taken of: the three plasmids AAV:ITR-U6-sgRNA(backbone)-pCBh-Cre-WPRE-hGHpA-ITR(Addgene, cat #60229), pHelper, and rAAV2-retro helper (addgene, cat #81070) for constructing a retrograding cre virus; the three plasmids pAAV-hChR2(H134R)-EYFP, pHelper, and pAAV-RC for constructing AAV-ChR2 virus; and the three plasmids AAV-DIO-hM3D(Gq)-mcherry(Addgene, cat #44361), pHelper, and pAAV-RC for constructing AAV-DIO-hM3Dq. The sets of the three plasmids were transduced into AAV-293 cells (packaging cell line) to produce the desired viruses.

Therefore, the method for constructing an animal model of obsessive-compulsive disorder may be achieved through a step of activating a neural circuit connecting between the basolateral amygdala (BLA) and the dorsomedial striatum (DMS) in an animal.

In the present disclosure, the activation of the neural circuit may be physical activation or chemical activation.

The physical activation may be carried out by injecting a virus carrying a channel rhodopsin gene into the BLA, and establishing an optical fiber at the DMS, and then emitting light to the DMS.

The chemical activation may be carried out by injecting a virus carrying DIO (Double-Floxed Inverted Open reading frame) and a chemogenetic protein gene into the BLA, injecting a retrograde virus carrying a Cre recombinant enzyme gene into the DMS, and applying a drug upon activation of the viruses.

As used herein, the term “chemogenetics”, also called chemobiology or chemogenomics, refers to an applied technology that helps understand the physiological function of regulating cell activity by applying a protein that responds to an unknown, low-molecular synthetic material. Various proteins including kinases, non-kinase enzymes, GPCR (G-protein-coupled receptors), and ligand-gated ion channel have been established in a chemogenetic manner. The most commonly used among various chemogenetically designed proteins are DREADDs (designer receptors exclusively activated by designer drugs) which are designed to decrease in reactivity to acetylcholine, but to increase in reactivity to new synthetic materials (e.g., clozapine-N-oxide) by partially mutating a wild-type muscarinic receptor.

As used herein, the term “chemogenetic protein” refers to a receptor protein designed in a chemogenetics manner to be activated exclusively by a specific drug.

In the present disclosure, examples of the chemogenetic protein include, but are not limited to, hM2Di, hM4Di, hM3Dq, and hM5Dq.

In the present disclosure, the gene encoding the hM3Dq protein may include the nucleotide sequence of SEQ ID NO: 1 and the gene encoding the hM4Di protein may include the nucleotide sequence of SEQ ID NO: 2.

In the present disclosure, the drug that activates the chemogenetic protein may be exemplified by clozapine N-oxide (CNO), clozapine, compound 21, and perlapine, but with no limitations thereto.

In the following Examples of the present disclosure, hM3Dq was used as a chemogenetic protein which was activated by clozapine N-oxide (CNO).

As used herein, the term “Double-Floxed Inverted Open reading frame” (DIO) refers to an open reading frame which can function as a LoxP site-based genetic switch and can change a turn-off state to a turn-on state of gene expression in the presence of Cre recombinase.

The DIO (Double-Floxed Inverted Open reading frame) may include a pair of LoxP sites and a pair of Lox2722 sites which are all recognized by Cre recombinase. For example, the DIO may include the LoxP sequence of SEQ ID NO: 3, the reverse LoxP sequence of SEQ ID NO: 4, the Lox2722 sequence of SEQ ID NO: 5, and the reverse Lox2722 sequence of SEQ ID NO: 6.

A gene encoding the Cre recombinase may include the nucleotide sequence of SEQ ID NO: 7.

In addition, the present disclosure provides an animal model of obsessive-compulsive disorder, constructed by the method.

The animal model of obsessive-compulsive disorder according to the present disclosure conducts all the compulsive behaviors including checking behavior, repetitive behavior, cleansing behavior, and hoarding behavior while becoming low in cognitive flexibility.

In the following Examples of the present disclosure, an animal model of obsessive-compulsive disorder was constructed by activating the BLA-DMS neural circuit in mice. As a result, the animal model was observed to remarkably frequently conduct a, checking behavior, repetitive nose poking, excessive grooming (cleansing behavior), and a behavior of storing feeds and toys (hoarding behavior) while exhibiting poor cognitive flexibility.

Also, the present disclosure provides a method for screening a candidate drug for prevention or treatment of obsessive-compulsive disorder, the method comprising the steps of: administering a candidate drug to the animal model of obsessive-compulsive disorder; and determining whether any of the obsessive-compulsive disorder-caused behaviors including checking behavior, repetitive behavior, cleaning behavior, and hoarding behavior is reduced.

As used herein, the term “administration” means introduction of a candidate drug into an animal model of obsessive-compulsive disorder with the aid of any suitable method. So long as it reaches a target tissue, any general route may be taken. A candidate drug may be administered orally, intraperitoneally, intravenously, intramuscularly, subcutaneously, intradermally, intranasally, intrapulmonarily, intrarectally, intrathecally, or intradurally.

A better understanding of the present disclosure may be obtained through the following examples, which are set forth to illustrate, but are not to be construed to limit the present disclosure.

### MODE FOR CARRYING OUT THE INVENTION

### Examples

1. Test Methods

Test Animal

Male (BL/6N) mice were purchased from Orient Bio (Seoul, Korea) and reared under a 12/12 hours light/dark cycle, with access to food and water ad libitum. All the animal tests were conducted according to the guideline approved by the Institute Animal Care and Use Committee (IACUC) of the Korea University.

Virus Construction

For virus construction, an AAV Helper-Free system (Agilent, cat #240071) was used. AAV-293 cells were seeded into seven 10-cm tissue culture plates and incubated at 37° C. in a 5% CO₂ incubator. After 2-3 days, transfection was conducted at 70-80% confluency. For ‘Retrograding cre virus’, the three plasmids: AAV:ITR-U6-sgRNA(backbone)-pCBh-Cre-WPRE-hGHpA-ITR (Addgene, cat #60229); pHelper; and rAAV2-retro helper (addgene, cat #81070) were each prepared at a concentration of 1 μg/μl in TE buffer, pH 7.5 and simultaneously transfected into the cells. For ‘AAV-ChR2 virus’ the three plasmids: pAAV-hChR2(H134R)-EYFP obtained by treating pAAV-EF1a-double floxed-hChR2(H134R)-EYFP (Addgene cat #20298) with Cre recombinase to remove the double floxed gene; pHelper; and pAAV-RC were employed. For ‘AAV-DIO-hM3Dq’ virus, the three plasmids: pAAV-hSyn-DIO-hM3D(Gq)-mcherry(Addgene, cat #44361); pHelper; and pAAV-RC were used.

The three plasmids were each added in an amount of 10 μl to a 15-ml conical tube and carefully mixed with 1 ml of 0.3M CaCl₂). The mixture was dropwise added to 1 ml of 2×HBS (Hepes Buffered Saline, pH 7.1) and then mixed carefully. Subsequently, 2.03 ml of the resulting solution was dropwise added to the cells incubated for 2-3 days and the cells were again input to the 37° C. incubator. After 6 hours, the culture medium was changed with a fresh one. Then, additional incubation for 66-72 hours in the 37° C. incubator allowed the production of viruses. Next, a 37° C. water bath and a dry ice-ethanol bath were prepared and the culture of the transfected cells was added to a 50-ml conical tube and sealed. The tube was placed for 10 minutes in the dry ice-ethanol bath to freeze the medium and cells. Thereafter, the completely frozen tube was thawed for 10 minutes in the water bath. If the tube was not completely thawed within 10 minutes, it was kept in the water bath to complete thawing. Then, the completely thawed tube was slightly agitated. This procedure was repeated a total of four times to lyse the cells and extract the viruses. Centrifugation at 10,000×g for 10 minutes precipitated cell debris. The supernatant was mixed with 40% PEG (polyethylene glycol) to prepare a 10% PEG solution. That is, 40% PEG was mixed with the viral solution at a ratio of 1:3 and incubated at 4° C. for two days. For enrichment of the viruses after two days, centrifugation for 30 minutes at 3,000 rpm and 4° C. was followed by decanting the supernatant. The pellet was suspended in 10 ml of ice-cold PBS and then centrifuged for 2 hours at 29,000 rpm and 4° C. The supernatant was removed and the pellet was resuspended in 1 ml of ice-cold PBS.

Stereotaxic Injection of Virus

For stereotaxic injection, mice at 8-9 weeks of age were used. Each mouse was anesthetized with ketamine (100 mg/kg, Yuhan)+Rompun (xylazine, 10 mg/kg, Bayer Korea). The head of the anesthetized mouse was fixed in a stereotaxic apparatus (David Kopf instrument, Tujunga, Calif., USA). At the predetermined coordinates, a hole was made through the skull using an electric drill. A glass micropipette was inserted to inject viruses with the aid of Nanoliter 2000 (World precision instrument, Sarasota, Fla., USA) injector (BLA coordinates: AP −1.5, ML ±3.1, and DV −4.8 from bregma, DMS coordinates: AP +1.1, ML ±1.1, and DV −3.0 from bregma). Each virus was slowly injected a total of 15 times at a dose of 9.2 nl per injection (23 nl/sec).

Optic cannulas including Ferrule (precision fiber products, MM-FER2007-304-2300) and Fiber (Thorlabs, FT200UMT) were used. The optical cannulas were cut (3 mm) to fit the depth of the DMS area, inserted, and fixed using dental cement (Poly-F). Then, the viruses were allowed to spread for three weeks before conducting a behavioral test.

Drug Treatment

A solution of clozapine N-oxide (CNO; Tocris, Bristol, United Kingdom) in saline was intraperitoneally injected to mice. Taking into account the time it takes for the drug to act, the experiment was started after about 30 minutes. The drug was injected at a dose of 1 mg/kg at the same time every day.

As a control for CNO, saline was used. Saline was also intraperitoneally injected at the same volume and the experiments were conducted in the same condition as for the CNO group.

Behavior Test

[Elevated Plus Maze (EPM) Test with Optogenetic Activation]

The EPM apparatus consisted of two open arms (68 cm×7 cm×0.5 cm) and two closed arms (68 cm×7 cm×17 cm). Each of the closed arms was enclosed by a 17-cm wall, and the EPM was elevated 57 cm from the ground. The mouse was placed in the center of the cross maze and the locomotion of the animal was monitored for a total of 15 minutes. An OFF phase and an ON phase were alternated every 3 minutes. As a light source, blue light (473 nm, 20 Hz, 10 mW, CNI laser, MBL-FN-473-150 mW) was employed, with a pulse generator (BNC model 575) for controlling the light source. Analysis was made using EthoVision XT 11.5 (Noldus, Wageningen, Netherlands).

[Hole Board Test]

A square acrylic plate with 9 holes (3 cm in diameter) was made to be 30 cm wide and 30 cm long. A home base was constructed with a material different from the acryl plate. Based on the home base, water was placed on the right end of the acrylic plate, and 10% sucrose was placed on the other side. Afterward, a mouse was placed in the home base and monitored for 1 hour under a video camera. Remaining amounts of sucrose and water were recorded after completion of the test.

In the present disclosure, mice were divided into two groups: saline-administered and CNO-administered groups and subjected to the test at the same time every day for 18 days. After a four-day learning period, the fourth day was used as the base. During this period, the mice located the water and 10% sucrose on the hole board. Saline and CNO were administered to each group for a total of 12 days from the 5th day to the 16^{th} day. On the 17^{th} day, it was observed whether the OCD phenomenon was maintained even in the absence of administration of saline and CNO. Finally, on the 18^{th} day, the mice were measured for cognitive flexibility after switching the positions of water and 10% sucrose. The video between the base and the 18^{th} day (base to day 14) was analyzed. The analysis items included checking behavior (repetitive checking), repetitive nose poking (repetitively inserting the nose into the hole), excessive grooming (excessive washing—hair trimming), and flexibility (cognitive flexibility).

As an analysis method, the video was analyzed with the naked eye through the blind test. Through the blind test, the objectivity of the experiment was secured by preventing the experimenter from knowing about the saline-administered group and the CNO-administered group. A measurement was made of: the frequency of going to water and sucrose for the checking behavior (repetitive checking); the number of actions of inserting the nose into each hole for repetitive nose poking (repeatedly inserting the nose into the hole); the total length of grooming (duration) and the total number of times (frequency) of the start and end of grooming for excessive grooming (excessive washing—hair trimming); and the number of times to go to water and sucrose where the positions of the 18^{th} day (day 14) were reversed, for the flexibility (cognitive flexibility).

Hoarding (storage disorder) was observed in the absence of administration of saline and CNO for a total of 16 hours from immediately after the end of the experiment on the 17^{th} (day 13) to the morning of the 18^{th} day (day 14).

[Hoarding Test]

A tool was manufactured by connecting two cages, with a passage in the middle thereof. The cages each have a dimension of width 25 cm×length 20 cm×height 12 cm (Jeung Do PNP). For the passage, a PVC cylinder with a dimension of diameter 5 cm and length 19 cm was used. A hole with a diameter of 5 cm was drilled 1 cm above the bottom of each cage, and a sill was installed on the PVC so that the length of the passage was 9.5 cm when a PVC cylinder was inserted into the holes. Mice were placed one per cage. In order for the mouse to recognize one cage as a home cage, beddings were placed on only one cage and the other cage was left in the default state, and the passageway to the opposite side of the home cage was blocked for one day. Then the next evening, the food in the home cage was removed, foods and toys were placed on the other cage, and the blocked passage was opened. At this time, water was placed in the home cage. Differences between the groups were compared in how many food pellets and toys were brought to the home cage on the morning of the last day of the experiment. Quantification was made of the brought weight for food and the brought number for toys. The toys used included a mixture of heart-shaped plastics, each with a diameter of about 0.5 cm and a weight of about 0.2 g, and square-shaped tiles each measuring a width of 0.5 cm×a length of 0.5 cm×a height of 0.1 cm and weighing about 0.9 g.

2. Test Results

Neural Circuit Connecting from the Basolateral Amygdala (BLA) to the Dorsomedial Striatum (DMS) was Identified.

Anterograde staining with AAV-tdTomato and retrograde staining with CTB (choleratoxin subunit b) demonstrated that the basolateral amygdala and the dorsomedial striatum in the brain are connected to each other.

As shown in FIG. 1, the BLA was stained red in the anterograde direction when AAV-tdTomato was injected thereto. At the site, a striatum area represented by the CPu (caudate putamen), especially the dorsomedial striatum (DMS) and the nucleus accumbens (NAc) were stained.

When CTB was injected targeting the dorsomedial striatum among the stained area, retrograde staining toward the basolateral amygdala was observed. Taken together, the staining data indicate that there is a circuit from the basolateral amygdala to the dorsomedial striatum.

The Role of the BLA-DMS Circuit was Identified Through Optogenetics Experiments.

AAV-ChR2 virus was injected into the BLA site, and an optical fiber was installed in the DMS site (see FIG. 2). Axon terminals projecting from the BLA express channel rhodopsin (ChR2). When exposed to blue light, ChR2 acts in the cell membrane to cause an influx of Na+ ions, activating neurons. While This technology (Optogenetics) was used to selectively activate the BLA-DMS circuit by applying blue light (473) specifically to the DMS among the sites receiving signals from the BLA, an elevated plus maze (EPM) test was conducted.

The EPM is a test measuring anxiety mainly in rodents such as mice. In the EPM, it is interpreted that the longer the time spent on the open arm, the lower the anxiety, and the longer the time spent on the closed arm, the higher the anxiety. As a result of observing the behavioral change of mice (BL6/N), the representative grooming behavior (hair trimming) among obsessive-compulsive symptoms increased rapidly immediately after receiving light. At the same time, it was seen that the rate of open arm duration in the EPM decreased while receiving light (see FIG. 3). This data suggests that activation of the BLA-DMS circuit may increase anxiety and further induce obsessive-compulsive symptoms.

Construction of Animal Model of Obsessive-Compulsive Disorder

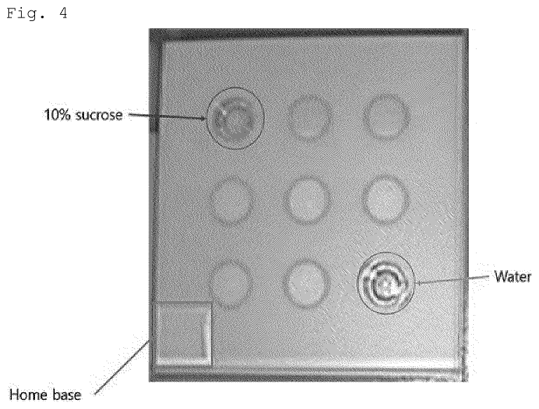

First, an animal model of obsessive-compulsive disorder was constructed using the BLA-DMS circuit, and a hole board test was designed to easily examine the expression of obsessive-compulsive disorder phenomena. In this regard, A square acrylic plate with 9 holes (3 cm in diameter) was made to be 30 cm wide and 30 cm long. A home base was constructed with a material different from the acryl plate. Based on the home base, water was placed on the right end of the acrylic plate, and 10% sucrose was placed on the other side (see FIG. 4).

For construction of an animal model of obsessive-compulsive disorder, a method for activating the BLA-DMS circuit in mice for a long term, but not temporarily, was designed (see FIG. 5).

AAV-retro Cre virus, which can make a retrograde infection, was injected to the DMS while AAV-DIO-hM3Dq, which specifically expresses Cre, was injected into the BLA. That is, advantage was taken of the principle that when the retrograde virus from the DMS expresses Cre in the neuron cell body (soma) of the BLA, the AAV-DIO-hM3Dq virus is induced to express hM3Dq thereat only in the presence of Cre. hM3Dq can activate neurons in cooperation with the membrane protein GPCR (G-protein-coupled receptors) upon stimulation with the drug CNO (clozapine n-oxide), whereby the specific activation of the BLA-DMS neural circuit can be achieved. After injecting different viruses into these two areas, it takes more than 4 weeks for the viruses to show their activities.

After 4 weeks, the learning process was performed for the first 4 days on the hole board without injection of CNO (clozapine n-oxide). During this process, the mice identify and remember the location of the water and 10% sucrose. The BLA-DMS circuit was continuously activated by injecting CNO (1 mg/kg) at the same time every day for 12 days, using the behavioral values of the 4^{th} day as a reference. For a control against CNO (clozapine n-oxide), saline was administered to the mice that had undergone the same process (injected with the same viruses). While a hole board test was conducted during this period, the saline-administered group and the CNO (clozapine n-oxide)-administered group of the mice were observed for changes in compulsive behaviors (1. checking behavior (repetitive checking), 2. repetitive nose poking (repetitively inserting the nose into the hole), 3. excessive grooming (excessive washing—hair trimming), and 4. flexibility (cognitive flexibility)). After one day, it was observed whether the compulsive behavior was maintained even without CNO (clozapine n-oxide) administration, and the following day, a hoarding (storage disorder) pattern was observed. Subsequently, the position of water and 10% sucrose was changed, and it was observed whether there was a difference in cognitive flexibility between the saline-administered group and the CNO-administered group was observed. This lack of cognitive flexibility is a major feature of the obsessive-compulsive disorder.

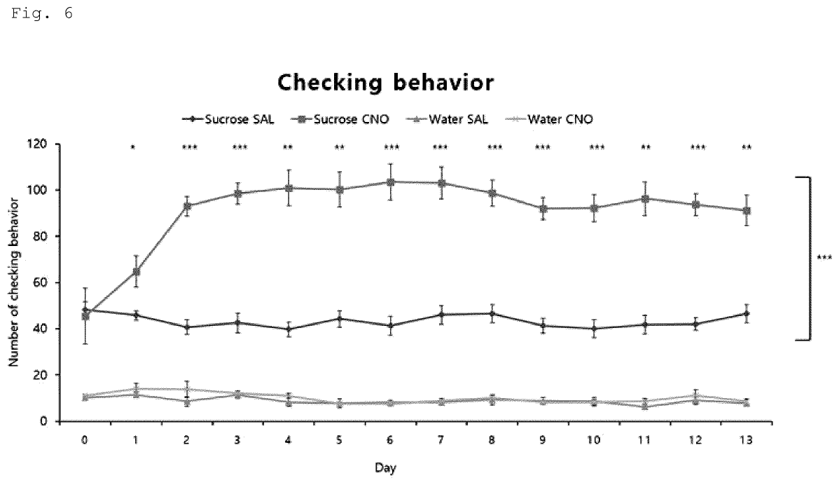

As shown in FIG. 6 depicting the result, there was no difference in the frequency of heading toward water in both the saline-administered group and the CNO (Clozapine n-oxide)-administered group, but there was a significant difference between the groups in the frequency of heading toward 10% sucrose (repeated 1-way ANOVA). In particular, the checking frequency of heading toward 10% sucrose in the CNO (chlorine n-oxide)-administered group was significantly increased, compared to the control.

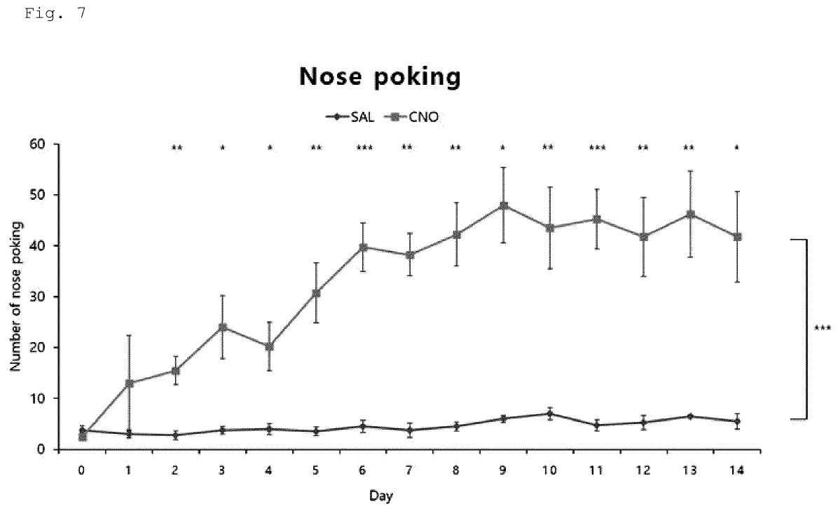

In addition, as shown in FIG. 7, it was confirmed that repetitive nose poking (the action of repeatedly inserting the nose into the hole) also showed a significant difference between the saline-administered group and the CNO (clozapine n-oxide)-administered group (repeated 1-way ANOVA). The CNO (Clozapine n-oxide)-administered group was observed to conduct nose poking at greatly increased frequencies.

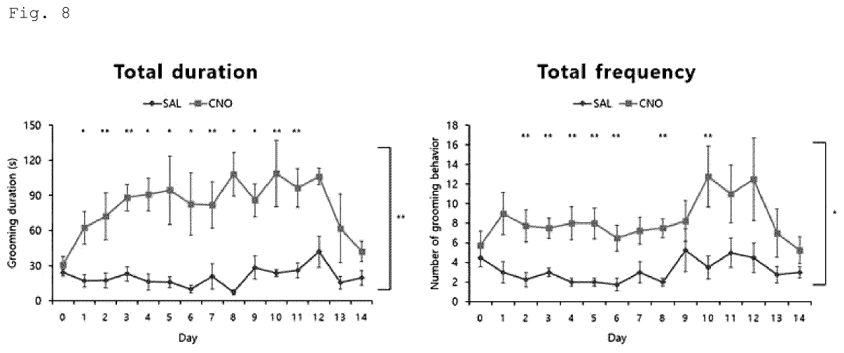

As shown in FIG. 8, both the grooming time and frequency showed significant differences between the saline-administered group and the CNO (Clozapine n-oxide)-administered group (Repeated 1-way ANOVA). Both the time and frequency were measured to be significantly increased in the CNO (clozapine n-oxide)-administered group, compared to the saline-administered group. For reference, grooming analyzed in this experiment is an action that corresponds to excessive hand washing, known as a representative symptom of obsessive-compulsive disorder.

Among others, data from checking and nose poking show that the obsessive-compulsory disorder was maintained even on day 13 when saline and clozapine n-oxide (CNO) were not administered.

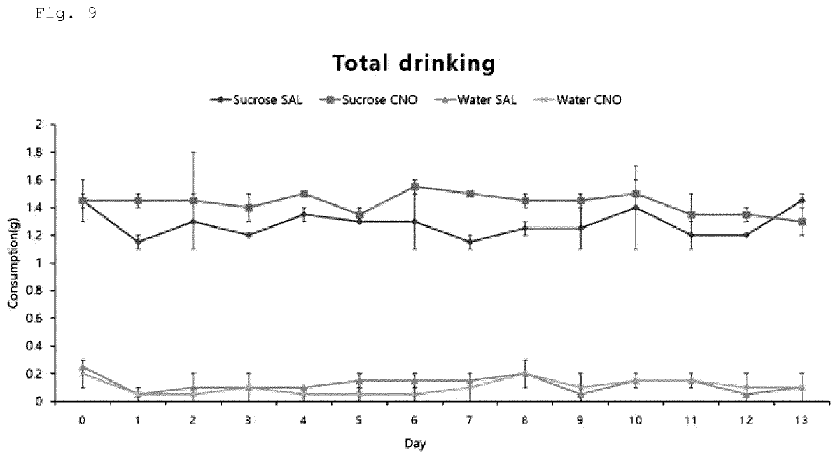

On the other hand, as shown in FIG. 9, there was no difference between the saline-administered group and the CNO (clozapine n-oxide)-administered group in the amount of 10% sucrose drank despite a significant difference in the checking frequency. These data demonstrate that the results of action conducted so far were not differences due to rewards, but repetitive behaviors due to obsessive-compulsive disorder.

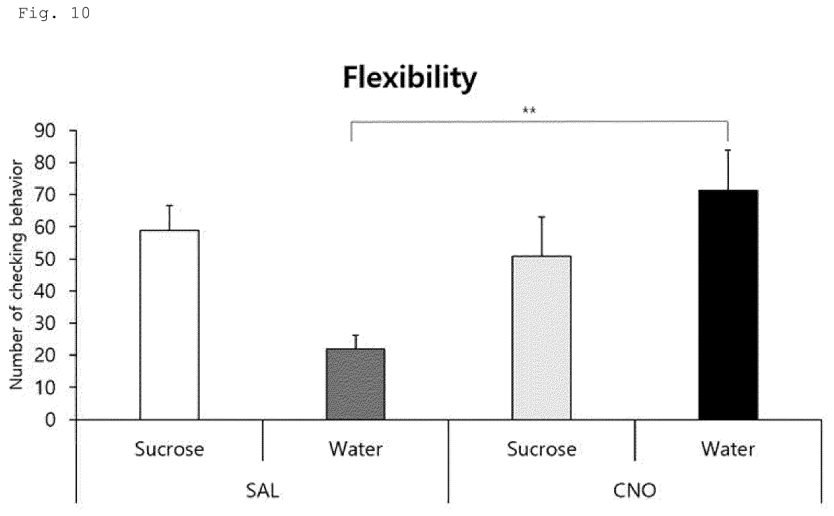

In addition, the results of the cognitive flexibility test performed on the last day of the experiment (day 14) confirmed, as shown in FIG. 10, that the flexibility was significantly lower in the CNO (Clozapine n-oxide)-administered group than in the saline-administered group (Student t-test). In the case of flexibility, the number of times to go to water and sucrose which had swapped positions on the 18^{th} day (day 14) was measured.

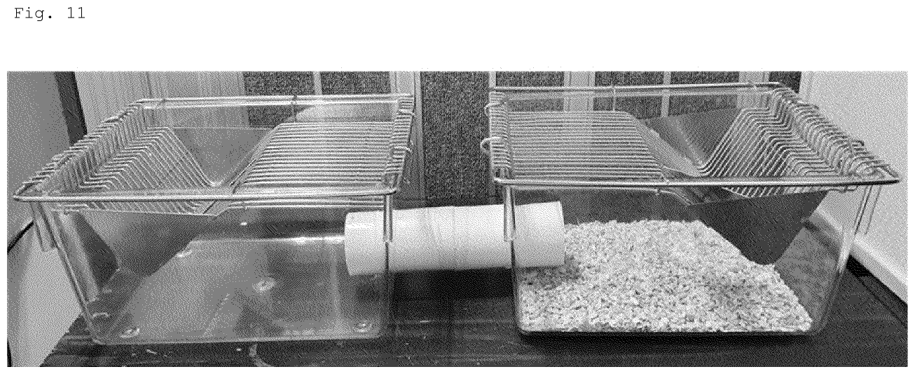

For use in identifying hoarding patterns, two cages were attached to each other with a central passageway (see FIG. 11). In order to allow the mice to recognize one case as a home case, the passageway to the opposite side were blocked for one day (day 12). In the evening of the next day (day 13), food and toys were placed on the opposite side and the blocked passage was opened. In this context, water was placed in the home cage. In the morning of the last day of the experiment (day 14), differences were observed between the saline-administered group and the CNO-administered group in terms of how many food pellets and toys were brought to the home cage.

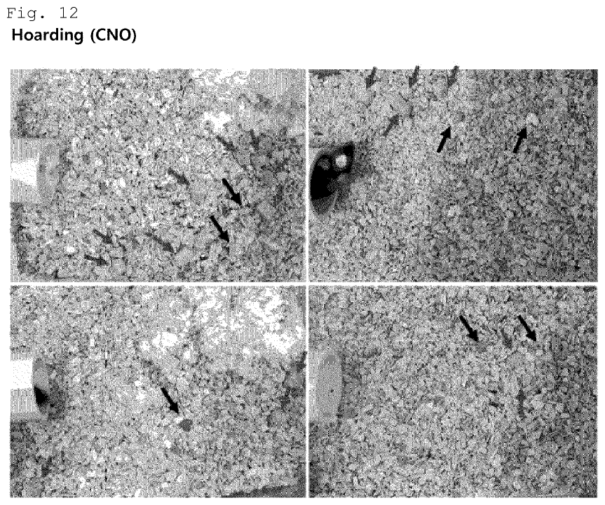

As shown in FIGS. 12 to 14, it was observed that hoarding of both food pellets and toys was significantly increased in the CNO (Clozapine n-oxide)-administered group compared to the saline-administered group. Particularly, toys, although unnecessary, were stored in a significantly large amount in the CNO (Clozapine n-oxide)-administered group, so it can be seen that hoarding of obsessive-compulsive disorder was implemented.

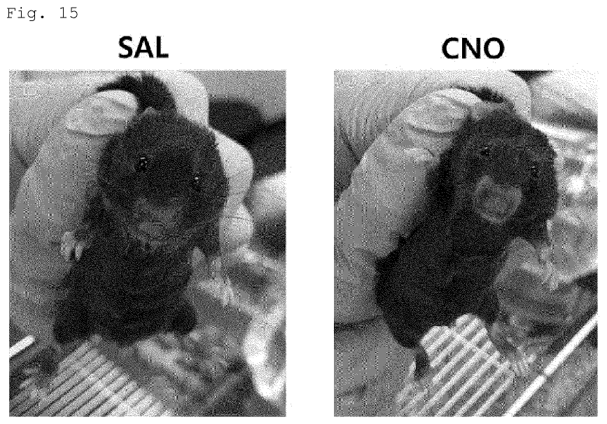

Additionally, after 20 days from the last day, it was observed that the hairs around the mouth of the Clozapine n-oxide (CNO)-administered mice fell out, compared to the saline-administered mice, indicating a phenotype caused by excessive grooming (see FIG. 15).

Although particular embodiments of the present disclosure have been described herein, it should be understood that the foregoing embodiments and advantages are merely examples and are not to be construed as limiting the present disclosure or the scope of the claims. Numerous other modifications and embodiments can be devised by those skilled in the art that will fall within the spirit and scope of the principles of this disclosure, and the present teaching can also be readily applied to other types of methods and apparatuses. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

### INDUSTRIAL APPLICABILITY

The animal in which the BLA-DMS neural circuit is activated according to the present invention can be usefully used as an animal model to study obsessive-compulsive disorder.

1. A method for constructing an animal model of obsessive-compulsive disorder, the method comprising a step of activating a neural circuit connecting from basolateral amygdala to dorsomedial striatum in animals exclusive of humans.

2. The method of claim 1, wherein the neural circuit is activated through physical activation or chemical activation.

3. The method of claim 2, wherein the physical activation is carried out by injecting a virus carrying a channel rhodopsin gene into a basolateral amygdala site, installing an optical fiber in a dorsomedial striatum site, and applying light to the dorsomedial striatum site to activate the neural circuit.

4. The method of claim 2, wherein the chemical activation is carried out by injecting a virus carrying a double-floxed inverted open reading frame (DIO) and a chemogenetic protein gene to a basolateral amygdala site, injecting a retrograde virus carrying a Cre recombinase gene to a dorsomedial striatum site, and then applying a drug upon activation of the viruses to active the neural circuit.

5. The method of claim 3, wherein the virus is adeno-associated virus (AAV).

6. The method of claim 4, wherein the chemogenetic protein is selected from the group consisting of hM2Di, hM4Di, hM3Dq, and hM5Dq.

7. The method of claim 4, wherein the drug activates a chemogenetic protein and is selected from the group consisting of clozapine N-oxide, clozapine, compound 21, and perlapine.

8. An animal model of obsessive-compulsive disorder, constructed by the method of claim 1.

9. The animal model of obsessive-compulsive disorder of claim 8, wherein the animal model exhibits all of checking behavior, repetitive behavior, cleaning behavior, and hoarding behavior.

10. A method for screening a candidate drug for prevention or treatment of obsessive-compulsive disorder, the method comprising the steps of:
administering a candidate drug to the animal model of obsessive-compulsive disorder of claim 8; and
determining whether any of obsessive-compulsive disorder-caused behaviors including checking behavior, repetitive behavior, cleaning behavior, and hoarding behavior is reduced.
